(19) United States
(12) Patent Application Publication
(10) Pub. No.: US 2024/0277066 A1
(43) Pub. Date: Aug. 22, 2024

(54) AEROSOL AMOUNT DETECTION METHOD AND NEBULIZER

(71) Applicant: HCMed Innovations Co., LTD., Taipei City (TW)

(72) Inventors: SHIH-CHAO LUO, New Taipei City (TW); CHIEN-SHEN TSAI, New Taipei City (TW); YUAN-MING HSU, New Taipei City (TW)

(21) Appl. No.: 18/433,674

(22) Filed: Feb. 6, 2024

Related U.S. Application Data
(60) Provisional application No. 63/445,138, filed on Feb. 13, 2023.

(30) Foreign Application Priority Data
Jan. 31, 2024 (CN) 202410137177.X

(51) Int. Cl. A24F 40/50; A24F 40/42

(52) U.S. Cl. CPC A24F 40/50; A24F 40/42

(57) ABSTRACT

An aerosol amount detection method and a nebulizer are provided. The aerosol amount detection method includes: continuously emitting, by a light detection circuit, a plurality of light signals to an aerosol path of the nebulizer for obtaining a plurality of light intensity values; calculating, by a control circuit, an aerosol amount index of the aerosol path according to the plurality of light intensity values; and outputting, by the control circuit, a warning signal when the aerosol amount index continues to be less than a stop threshold for a first check time.

### CROSS-REFERENCE TO RELATED PATENT APPLICATION

This application claims the benefit of priorities to China Patent Application No. 202410137177.X, filed on Jan. 31, 2024 and U.S. Provisional Patent Application Ser. No. 63/445,138, filed on Feb. 13, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an aerosol amount detection method and a nebulizer, and more particularly to a method for detecting an aerosol amount inside a nebulizer and the nebulizer.

### BACKGROUND OF THE DISCLOSURE

A nebulizing module of a nebulizer vibrates a medical liquid in a medicine cup to form an aerosol, and then a user can inhale the aerosol for treatment. When the medical liquid in the medicine cup is exhausted, and the nebulizing module continues to vibrate, the nebulizing module may be damaged. Such damage affects a particle size and stability of the aerosol in the subsequent treatment, thereby adversely affecting a therapeutic effect.

Currently, a method for avoiding damage to the nebulizing module is to detect a liquid level of the medical liquid in the medicine cup through a detection electrode of the medicine cup. When the liquid level is lower than a predetermined height, the nebulizing module stops vibrating. However, some types of the medical liquid are prone to foam during the vibration process. Due to the foam in the medicine cup, the detection electrode may fail to receive a correct liquid level. As a result, the nebulizing module continues to vibrate when the medical liquid is exhausted, such that the nebulizing module is damaged.

### SUMMARY OF THE DISCLOSURE

In response to the above-referenced technical inadequacy, the present disclosure provides an aerosol amount detection method and a nebulizer.

In order to solve the above-mentioned problem, one of the technical aspects adopted by the present disclosure is to provide an aerosol amount detection method. The aerosol amount detection method is adapted to a nebulizer, and includes: continuously emitting, by a light detection circuit, a plurality of light signals to an aerosol path of the nebulizer for obtaining a plurality of light intensity values; calculating, by a control circuit, an aerosol amount index of the aerosol path according to the plurality of light intensity values; and outputting, by the control circuit, a warning signal when the aerosol amount index continues to be less than a stop threshold for a first check time.

In order to solve the above-mentioned problem, another one of the technical aspects adopted by the present disclosure is to provide a nebulizer. The nebulizer includes a cup, a nebulizing module, a host, a light detection circuit, and a control circuit. The cup is configured to store a medical liquid. The nebulizing module is connected to the cup, wherein the nebulizing module is configured to convert the medical liquid into an aerosol and spread the aerosol to an aerosol path. The host is connected to the cup. The light detection circuit is disposed inside the host. The control circuit is disposed inside the host and connected to the light detection circuit. The light detection circuit is configured to continuously emit a plurality of light signals to the aerosol path for obtaining a plurality of light intensity values. The control circuit is configured to calculate an aerosol amount index of the aerosol path according to the plurality of light intensity values. When the control circuit determines that the aerosol amount index continues to be less than a stop threshold for a first check time, the control circuit outputs a warning signal.

Therefore, in the aerosol amount detection method and the nebulizer provided by the present disclosure, an aerosol amount of the nebulizer can be accurately detected, and an amount of the medical liquid in the nebulizer can be determined based on the aerosol amount. In this way, the probability of the nebulizing module continuing to vibrate when the medical liquid is exhausted can be reduced.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic perspective view of a nebulizer according to one embodiment of the present disclosure;

FIG. 2 is a schematic cross-sectional view taken along line II-II of FIG. 1;

FIG. 3 is a functional block diagram of the nebulizer according to a first embodiment of the present disclosure;

FIG. 4 is a flowchart of a method for establishing a nebulization stopping condition of the nebulizer according to the present disclosure;

FIG. 5 is a flowchart of an aerosol amount detection method adapted to the nebulizer of FIG. 3;

FIG. 6 is a functional block diagram of the nebulizer according to a second embodiment of the present disclosure;

FIG. 7 is a flowchart of the aerosol amount detection method adapted to the nebulizer of FIG. 6;

FIG. 8 is a timing diagram of an aerosol amount index of the nebulizer according to one embodiment of the present disclosure;

FIG. 9 is a timing diagram of a warning signal of the nebulizer according to one embodiment of the present disclosure;

FIG. 10 is a timing diagram of an activating signal of the nebulizer according to one embodiment of the present disclosure; and

FIG. 11 is a timing diagram of a stop signal of the nebulizer according to one embodiment of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

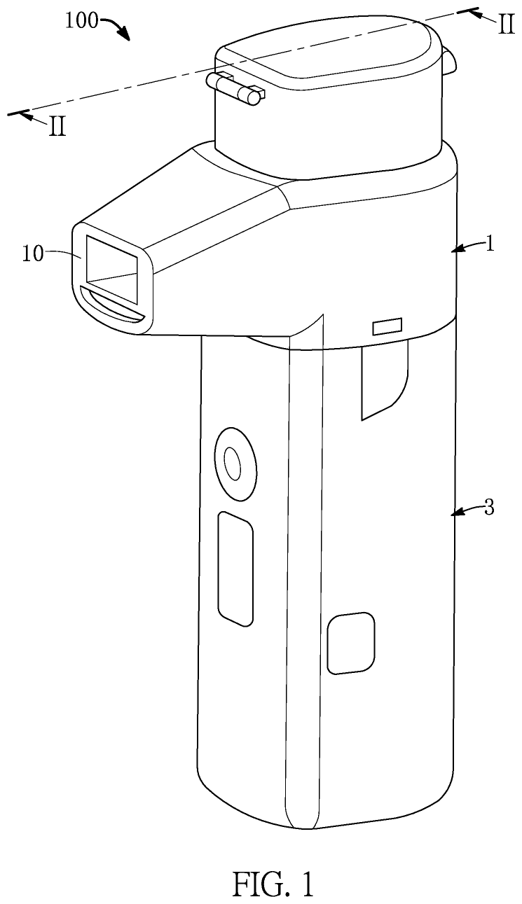

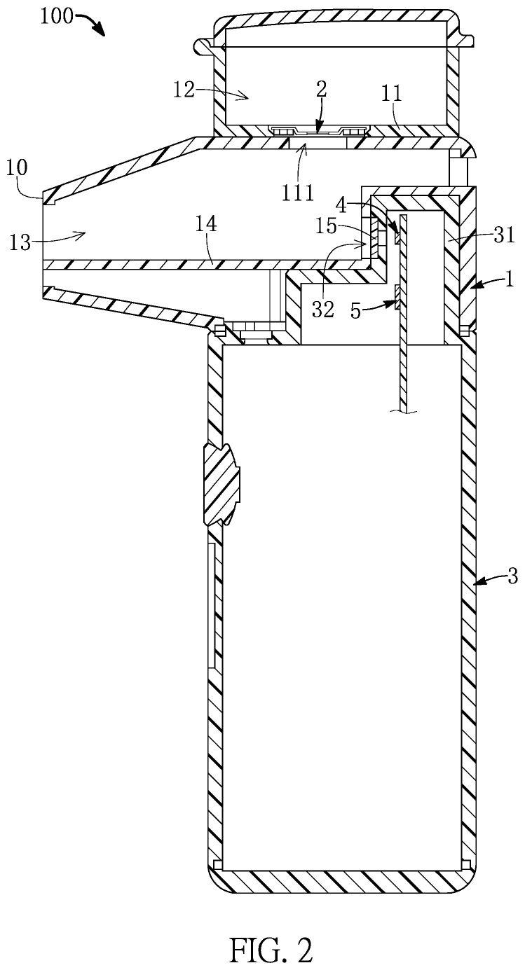

FIG. 1 is a schematic perspective view of a nebulizer according to one embodiment of the present disclosure, and FIG. 2 is a schematic cross-sectional view taken along line II-II of FIG. 1. Referring to FIGS. 1 and 2, a nebulizer 100 includes a cup 1, a nebulizing module 2, a host 3, a light detection circuit 4, and a control circuit 5. The cup 1 is movably connected to the host 3, the light detection circuit 4 and the control circuit 5 are arranged inside the host 3, and the control circuit 5 is electrically connected to the light detection circuit 4.

The cup 1 is provided with a nozzle 10 and a partition wall 11, and the partition wall 11 divides an inside of the cup 1 into a first chamber 12 and a second chamber 13. The first chamber 12 is used to store a medical liquid (not shown). The nebulizing module 2 is disposed on the partition wall 11. The partition wall 11 has a first through hole 111, and the first through hole 111 is located below the nebulizing module 2.

When a user inhales the air of the second chamber 13 through the nozzle 10, a pressure in the second chamber 13 will form a negative pressure as compared to an external atmospheric pressure. A bottom 14 of the cup 1 may be provided with a pressure sensor (not shown). When the pressure sensor detects the negative pressure, the pressure sensor will send a detection signal to the control circuit 5. Then, the control circuit 5 drives the nebulizing module 2 to convert the medical liquid in the first chamber 12 into an aerosol (not shown) according to the detection signal, and spread the aerosol into the second chamber 13 through the first through hole 111. The user inhales the aerosol in the second chamber 13 through the nozzle 10. Therefore, in this embodiment, an aerosol path is located in the second chamber 13. However, the aerosol path of the present disclosure is not limited thereto.

In one embodiment, the bottom 14 of the cup 1 has a concave stepped structure, and a joint surface 31 of the host 3 has a corresponding convex stepped structure. The light detection circuit 4 is disposed inside the convex stepped structure. The light detection circuit 4 emits a first light L1 to the second chamber 13 of the cup 1 through a second through hole 32 of the joint surface 31 of the host 3, so as to detect an aerosol amount in the second chamber 13. In a use environment, when the aerosol amount in the second chamber 13 is too much, the aerosol in the second chamber 13 may be condensed into water droplets. A light emitting surface of the light detection circuit 4 is located on a side vertical surface of the second chamber 13. As such, even if the aerosol condenses into the water droplets, the water droplets fall along the side vertical surface due to gravity, and do not accumulate on a light output surface of the light detection circuit 4, thereby not affecting an incidence of the first light L1. In an exemplary embodiment, a transparent wall 15 is provided between the second through hole 32 and the light detection circuit 4, and the second through hole 32 can also be appropriately filled with a translucent or transparent material. The translucent or transparent material not only allows light to pass through, but also prevents fog or moisture from penetrating into the host 3, so as to protect the light detection circuit 4 inside the host 3.

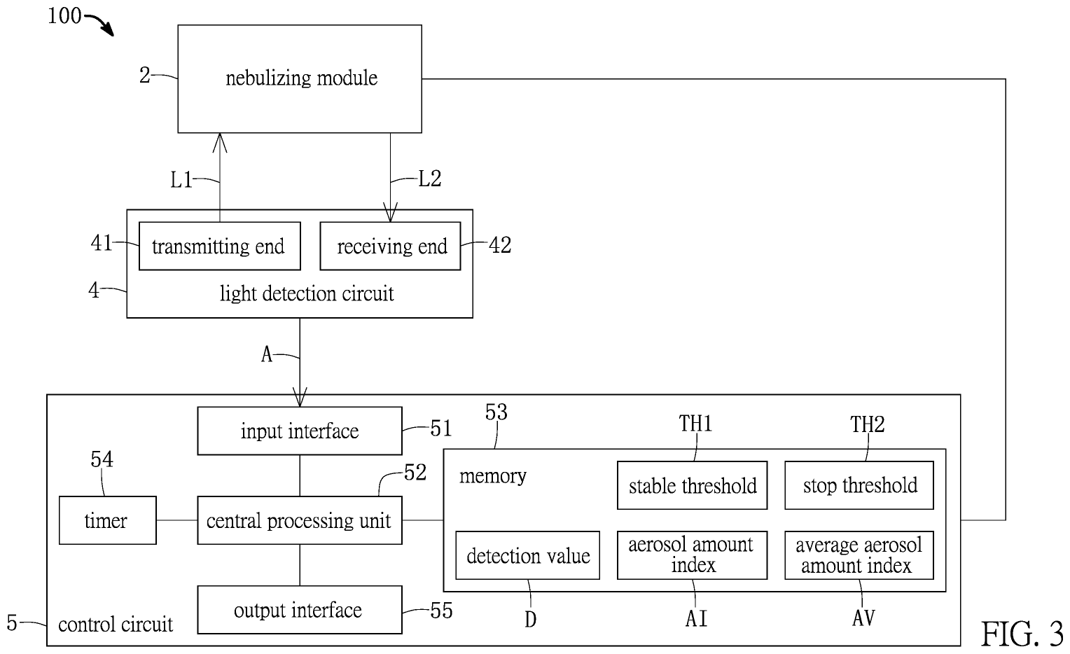

FIG. 3 is a functional block diagram of the nebulizer according to a first embodiment of the present disclosure. Referring to FIG. 2 and FIG. 3, the nebulizer 100 includes the nebulizing module 2, the light detection circuit 4, and the control circuit 5. The light detection circuit 4 is a light intensity detection device, and the light detection circuit 4 includes a transmitting end 41 and a receiving end 42. The transmitting end 41 emits a light signal (the first light L1) according to a fixed frequency, and the receiving end 42 is used to receive a reflected light signal (a second light L2) from the aerosol path. When the receiving end 42 receives the reflected light signal (the second light L2), the light detection circuit 4 obtains a light intensity value A of the reflected light signal and transmits the light intensity value A to the control circuit 5.

The control circuit 5 includes an input interface 51, a central processing unit 52, a memory 53, a timer 54, and an output interface 55. The central processing unit 52 is electrically connected to the input interface 51, the memory 53, the timer 54, and the output interface 55. The input interface 51 is used to receive the light intensity value A from the light detection circuit 4 and transmit the light intensity value A to the memory 53. The memory 53 stores a conversion algorithm for converting the light intensity value A into a detection value D. When the central processing unit 52 correspondingly converts a plurality of light intensity values A into a plurality of detection values D through the conversion algorithm, the plurality of detection values D can also be stored in the memory 53. When a number of the plurality of detection values D stored in the memory 53 reaches a preset sample number, the central processing unit 52 further calculates an average value of the plurality of detection values D. The average value calculated by the central processing unit 52 is an aerosol amount index AI of the aerosol path. In other embodiments, the preset sample number may be, for example, 10, but the present disclosure is not limited thereto.

It should be noted that, in the present disclosure, the light intensity value A is not directly taken as the aerosol amount index AI of the aerosol path due to significant changes of the light intensity value A. After the plurality of light intensity values A are converted into the plurality of detection values D, and the average value of the plurality of detection values D is calculated, using said average value as the aerosol amount index AI is more consistent with an actual use condition.

In another embodiment, the light detection circuit 4 can also calculate based on the light intensity value A of the reflected light signal, so as to obtain other aerosol parameters in the second chamber 13 (such as an aerosol particle size or an aerosol concentration). The above-mentioned aerosol parameters can be further adjusted by controlling a vibration frequency of the nebulizing module 2.

The memory 53 further stores a stable threshold TH1, a stop threshold TH2, a first check time, and a second check time. The central processing unit 52 determines whether or not the aerosol amount index AI of the aerosol path continues to be less than the stop threshold TH2 for the first check time according to a counting time of the timer 54. When the central processing unit 52 determines that the aerosol amount index AI of the aerosol path continues to be less than the stop threshold TH2 for the first check time, the central processing unit 52 outputs a warning signal to the nebulizing module 2 through the output interface 55, and notifies the nebulizing module 2 to prepare for stopping a nebulizing operation. After the warning signal continues for the second check time, the central processing unit 52 outputs a stop signal to the nebulizing module 2 through the output interface 55 for stopping the nebulizing operation of the nebulizing module 2.

Specifically, under a specific use condition, even if there is still enough medical liquid in the nebulizer 100, the nebulizer 100 may still be in an abnormal operation or an abnormal treatment position (such as being too tilted) at a certain time point. As a result, the nebulizing module 2 is not in contact with the medical liquid and cannot produce the aerosol, thereby causing the control circuit 5 to misjudge the aerosol amount index AI of the aerosol path to be less than the stop threshold TH2. Therefore, the purpose of setting the first check time is to reduce the probability of the control circuit 5 misjudging the aerosol amount index AI.

In order to reduce the time during which the nebulizer 100 continues to vibrate when the medical liquid is exhausted, a time point at which the medical liquid is exhausted needs to be accurately determined. Hence, it is necessary to establish an appropriate nebulization stopping condition of the nebulizer.

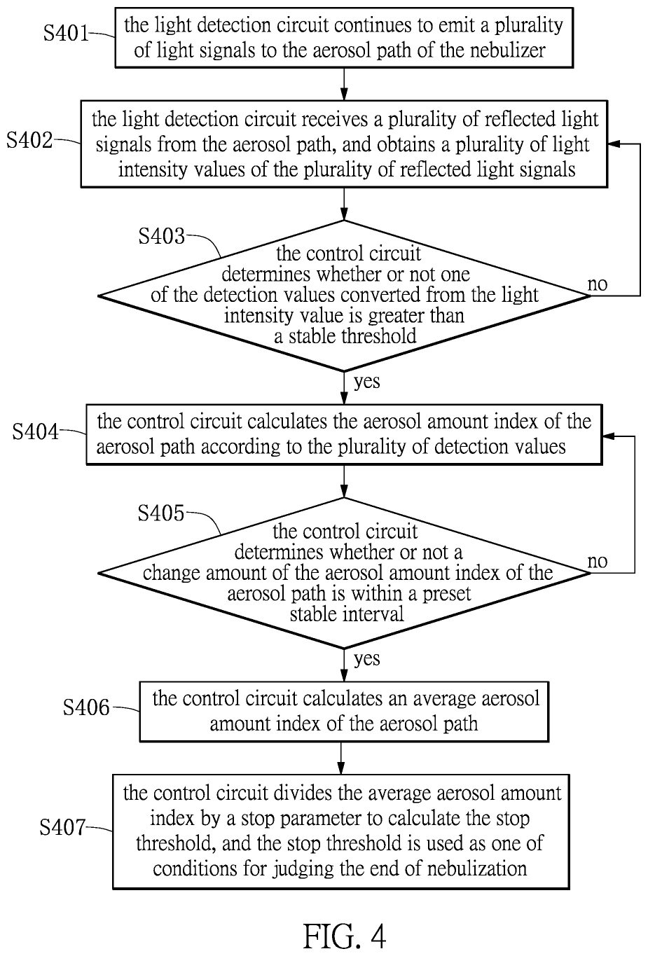

FIG. 4 is a flowchart of a method for establishing a nebulization stopping condition of the nebulizer according to the present disclosure. Referring to FIG. 4, in step S401, the light detection circuit 4 continues to emit a plurality of light signals to the aerosol path of the nebulizer 100. In this way, when aerosol particles exist in the aerosol path, one part of the light signals emitted by the light detection circuit 4 may hit the aerosol particles of the aerosol path and be reflected, and the other part of the light signals emitted by the light detection circuit 4 and not hitting the aerosol particles continues to travel along a light path.

In step S402, the light detection circuit 4 receives a plurality of reflected light signals from the aerosol path, and obtains a plurality of light intensity values A of the plurality of reflected light signals. Specifically, in step S401, when a part of the light signal emitted by the light detection circuit 4 hits the aerosol particles located in the aerosol path and is reflected, said part of the light signal can be defined as the reflected light signal. Since the light signal emitted by the light detection circuit 4 is only partially reflected, and the intensity of the reflected light signal may also be affected by scattering and other factors before being reflected to the light detection circuit 4, the intensity of the reflected light signal received by the light detection circuit 4 is less than the intensity of the light signal originally emitted by the light detection circuit 4.

In step S403, the control circuit 5 determines whether or not one of the detection values D converted from the light intensity value A is greater than a stable threshold TH1. Specifically, when the light detection circuit 4 obtains and transmits the light intensity value A of the reflected light signal to the control circuit 5, the control circuit 5 converts the light intensity value A into the detection value D according to the conversion algorithm. When the control circuit 5 determines that the detection value D is greater than the stable threshold TH1, step S403 is followed by step S404. When the control circuit 5 determines that the detection value D is not greater than the stable threshold TH1, the method returns to step S402.

It should be noted that the stable threshold TH1 can be a preset value, and can be regarded as a starting condition of a stable nebulizing operation. However, in other embodiments, 15% of a maximum value of the detection value D obtained in the previous treatment can also be regarded as the stable threshold TH1 of the present treatment, but the present disclosure is not limited thereto.

In step S404, the control circuit 5 calculates the aerosol amount index AI of the aerosol path according to the plurality of detection values D. Specifically, when the control circuit 5 determines that the detection value D is greater than the stable threshold TH1 and the number of the detection values D reaches the preset sample number, the control circuit 5 calculates an average value of the detection values D. The average value of the detection values D is the aerosol amount index AI of the aerosol path.

In step S405, the control circuit 5 determines whether or not a change amount of the aerosol amount index AI of the aerosol path is within a preset stable interval (e.g., −3% to 3%). When the control circuit 5 determines that the change amount of the aerosol amount index AI of the aerosol path is within the preset stable interval, it means that the aerosol amount index AI of the aerosol path is in a stable state, and step S405 is followed by step S406. When the control circuit 5 determines that the change amount of the aerosol amount index AI of the aerosol path is not within the preset stable interval, it means that the aerosol amount index AI of the aerosol path is not in the stable state, and the method returns to step S404.

Specifically, the change amount of the aerosol amount index AI is a percentage of a difference between the present aerosol amount index AI and the previous aerosol amount index AI relative to the previous aerosol amount index AI. The above-mentioned stable interval is preset in the conversion algorithm of the memory 53.

In step S406, when the aerosol amount index AI of the aerosol path is in the stable state, the control circuit 5 calculates an average aerosol amount index AV of the aerosol path. For example, the time interval covered by the stable state of the aerosol amount index AI of the aerosol path is 20 seconds. The average aerosol amount index AV can be calculated according to the aerosol amount indexes AI during the last 10 seconds of the time interval.

In step S407, the control circuit 5 divides the average aerosol amount index AV by a stop parameter to calculate the stop threshold TH2, and the stop threshold TH2 is used as one of conditions for judging the end of nebulization. For example, when the stop parameter is 10, it means that 10% of the average aerosol amount index AV is the stop threshold TH2. It should be noted that the setting of the stop parameter is related to the type of the medical liquid in the cup 1. Therefore, different stop parameters are set in response to different types of the medical liquid.

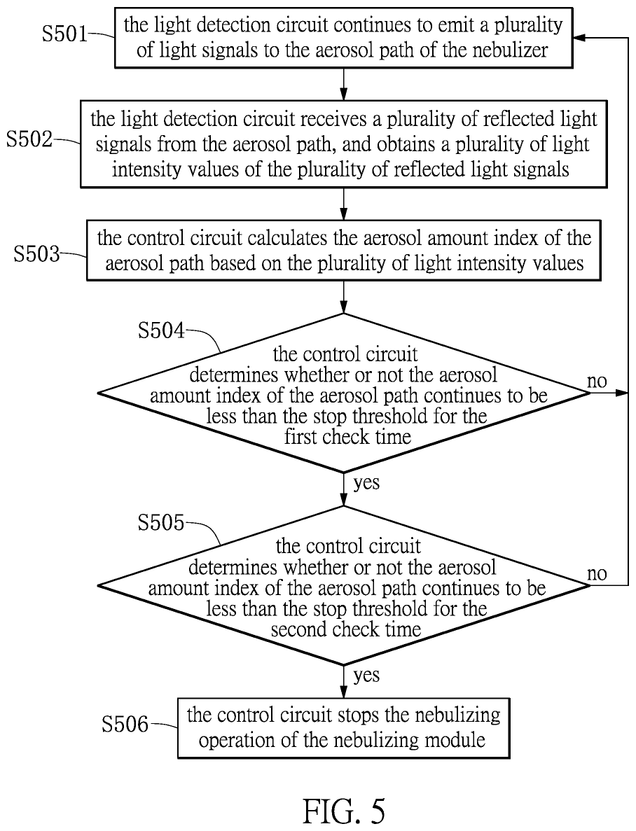

FIG. 5 is a flowchart of an aerosol amount detection method adapted to the nebulizer of FIG. 3. Referring to FIG. 5, in step S501, the light detection circuit 4 continues to emit a plurality of light signals to the aerosol path of the nebulizer 100.

In step S502, the light detection circuit 4 receives a plurality of reflected light signals from the aerosol path, and obtains a plurality of light intensity values A of the plurality of reflected light signals.

In step S503, the control circuit 5 calculates the aerosol amount index of the aerosol path based on the plurality of light intensity values A. Specifically, the control circuit 5 converts the plurality of light intensity values A of the plurality of reflected light signals received by the light detection circuit 4 into the plurality of detection values D. When a number of the plurality of detection values D reaches a preset sample number, the control circuit 5 calculates an average value of the plurality of detection values D, and the average value acts as the aerosol amount index AI.

In step S504, the control circuit 5 determines whether or not the aerosol amount index AI of the aerosol path continues to be less than the stop threshold TH2 for the first check time. Specifically, the aerosol amount index AI cannot be less than the stop threshold TH2 only at a certain time point. In order to truly represent the aerosol concentration of the aerosol path, the aerosol amount index AI must continue to be less than the stop threshold TH2 for a time period. When the control circuit 5 determines that the aerosol amount index AI of the aerosol path is less than the stop threshold TH2, the control circuit 5 can selectively output a warning signal to the nebulizing module 2, and notify the nebulizing module 2 to prepare for stopping a nebulizing operation.

When the control circuit 5 determines that the aerosol amount index AI of the aerosol path continues to be less than the stop threshold TH2 for the first check time, step S504 is followed by step S505. In other words, when an accumulated time during which the aerosol amount index AI is less than the stop threshold TH2 does not reach the first check time, the nebulizing module 2 still continues to operate.

When the control circuit 5 determines that the aerosol amount index AI of the aerosol path is not less than the stop threshold TH2 or a time period of being less than the stop threshold TH2 does not reach the first check time, the method returns to step S501.

In step S505, the control circuit 5 determines whether or not the aerosol amount index AI of the aerosol path continues to be less than the stop threshold TH2 for the second check time. In other words, only when the accumulated time during which the aerosol amount index AI is less than the stop threshold TH2 reaches the second check time, it is indicated that no aerosol exists in the aerosol path, and the medical liquid is exhausted.

When the control circuit 5 determines that the aerosol amount index AI of the aerosol path continues to be less than the stop threshold TH2 for the second check time, step S505 is followed by step S506.

When the control circuit 5 determines that the aerosol amount index AI of the aerosol path is not less than the stop threshold TH2 or the time period of being less than the stop threshold TH2 does not reach the second check time, the method returns to step S501.

In step S506, the control circuit 5 stops the nebulizing operation of the nebulizing module 2.

The reason why the nebulizing module 2 stops the nebulizing operation only after the warning signal continues for the second check time is to give the user of the nebulizer 100 a little buffer time to inhale the aerosol.

The above-mentioned aerosol amount detection method of the nebulizer can accurately monitor exhaustion of the medical liquid of the nebulizer, and can immediately and proactively stop the nebulizing operation of the nebulizing module.

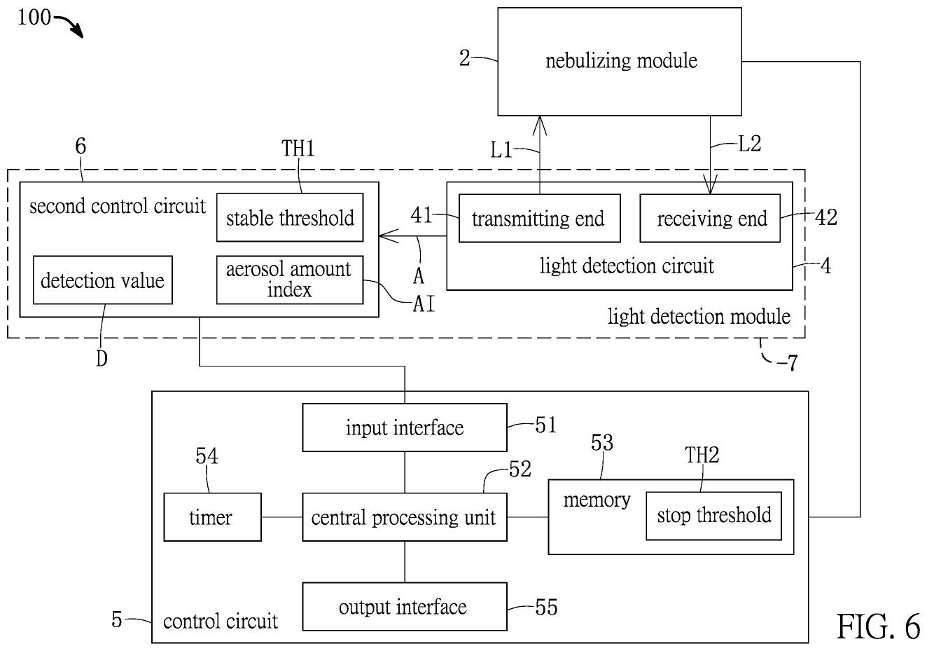

FIG. 6 is a functional block diagram of the nebulizer according to a second embodiment of the present disclosure. A difference between the second embodiment and the first embodiment is that the nebulizer 100 further includes a second control circuit 6. The second control circuit 6 and the light detection circuit 4 form a light detection module 7, and the second control circuit 6 is correspondingly and electrically connected to the control circuit 5 and the light detection circuit 4.

Each time the light detection circuit 4 obtains the light intensity value A of the reflected light signal, the light detection circuit 4 transmits the light intensity value A to the second control circuit 6. When the second control circuit 6 receives the light intensity value A from the light detection circuit 4, the second control circuit 6 converts the light intensity value A into the detection value D, and stores the detection value D in a memory of the second control circuit 6. When the number of the detection values D stored in the memory of the second control circuit 6 reaches the preset sample number of the conversion algorithm, the second control circuit 6 calculates an average value of the plurality of detection values D, and the average value acts as the aerosol amount index AI of the aerosol path. Then, the second control circuit 6 transmits the aerosol amount index AI to the control circuit 5. When the control circuit 5 determines that the aerosol amount index AI of the aerosol path continues to be less than the stop threshold TH2 for the first check time, the control circuit 5 outputs a warning signal to the nebulizing module 2 and notifies the nebulizing module 2 to prepare for stopping the nebulizing operation. After the warning signal continues for the second check time, the central processing unit 52 of the control circuit 5 outputs a stop signal to the nebulizing module 2 through the output interface 55 for stopping the nebulizing operation of the nebulizing module 2.

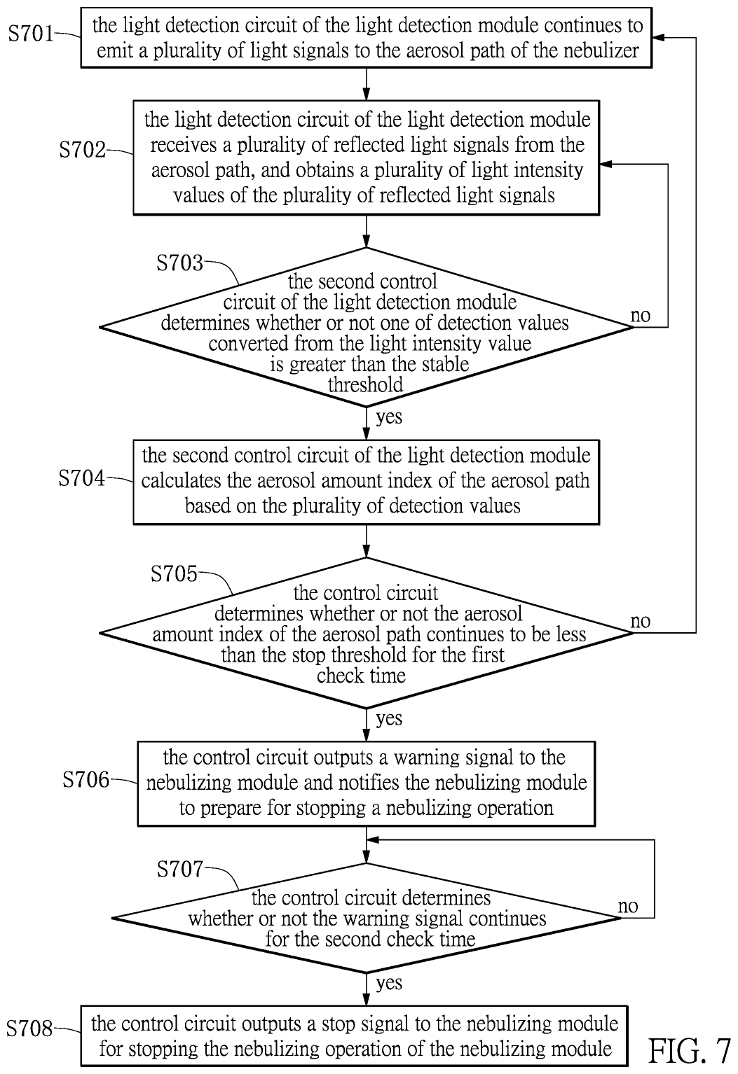

FIG. 7 is a flowchart of the aerosol amount detection method adapted to the nebulizer of FIG. 6.

In step S701, the light detection circuit 4 of the light detection module 7 continues to emit a plurality of light signals to the aerosol path of the nebulizer 100.

In step S702, the light detection circuit 4 of the light detection module 7 receives a plurality of reflected light signals from the aerosol path, and obtains a plurality of light intensity values A of the plurality of reflected light signals.

In step S703, the second control circuit 6 of the light detection module 7 determines whether or not one of detection values D converted from the light intensity value A is greater than the stable threshold TH1.

When the second control circuit 6 determines that the detection value D is greater than the stable threshold TH1, step S703 is followed by step S704. When the second control circuit 6 determines that the detection value D is not greater than the stable threshold TH1, the method returns to step S702.

In step S704, the second control circuit 6 of the light detection module 7 calculates the aerosol amount index AI of the aerosol path based on the plurality of detection values D.

In step S705, the control circuit 5 determines whether or not the aerosol amount index AI of the aerosol path continues to be less than the stop threshold TH2 for the first check time. When the control circuit 5 determines that the aerosol amount index AI of the aerosol path continues to be less than the stop threshold TH2 for the first check time, step S705 is followed by step S706. Conversely, when the control circuit 5 determines that the aerosol amount index AI of the aerosol path is not less than the stop threshold TH2 or a time period of being less than the stop threshold TH2 does not reach the first check time, the method returns to step S701.

In step S706, the control circuit 5 outputs a warning signal to the nebulizing module 2 and notifies the nebulizing module 2 to prepare for stopping a nebulizing operation.

In step S707, the control circuit 5 determines whether or not the warning signal continues for the second check time. When the control circuit 5 determines that the warning signal continues for the second check time, step S707 is followed by step S708. When the control circuit 5 determines that the warning signal does not continue for the second check time, step S707 is re-executed.

In step S708, the control circuit 5 outputs a stop signal to the nebulizing module 2 for stopping the nebulizing operation of the nebulizing module 2.

In the second embodiment of the aerosol amount detection method, since the second control circuit 6 is responsible for the calculation of converting the light intensity value A into the aerosol amount index AI, the computational load of the control circuit 5 is reduced. Furthermore, since the nebulizer 100 of the second embodiment is configured with two control circuits, even if the algorithm of the nebulizer 100 is upgraded in the future, the aerosol amount index AI can still be calculated in a timely manner.

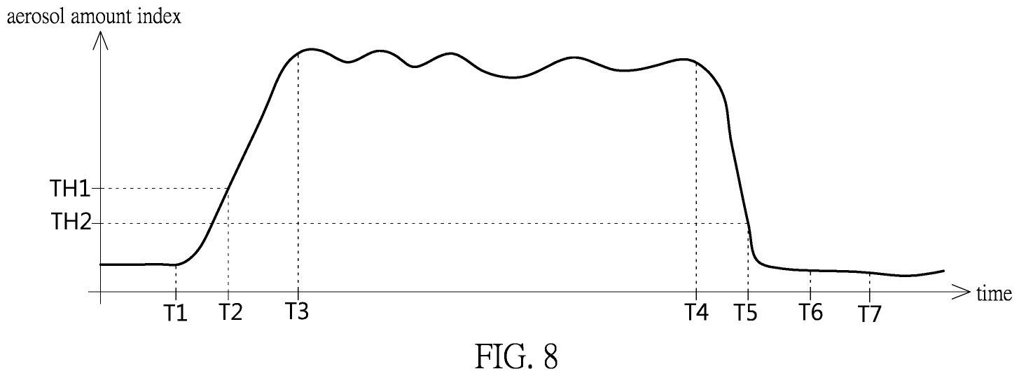

FIG. 8 is a timing diagram of an aerosol amount index of the nebulizer according to one embodiment of the present disclosure. Referring to FIG. 8, the nebulizing module 2 starts nebulizing the medical liquid at a first time point T1. At a second time point T2, the control circuit 5 determines that the aerosol amount index AI of the aerosol path reaches the stable threshold TH1, which indicates that the nebulizer 100 has reached the condition for starting the nebulizing operation (step S403). Between a third time point T3 and a fourth time point T4, the control circuit 5 determines that the change amount of the aerosol amount index AI of the aerosol path is within the preset stable interval, which indicates that the aerosol amount index AI of the aerosol path AI is in the stable state (steps S404 to S406). At a fifth time point T5, the control circuit 5 determines that the aerosol amount index of the aerosol path is less than the stop threshold TH2, which indicates that the nebulizing module 2 can be notified to prepare for stopping the nebulizing operation (step S407).

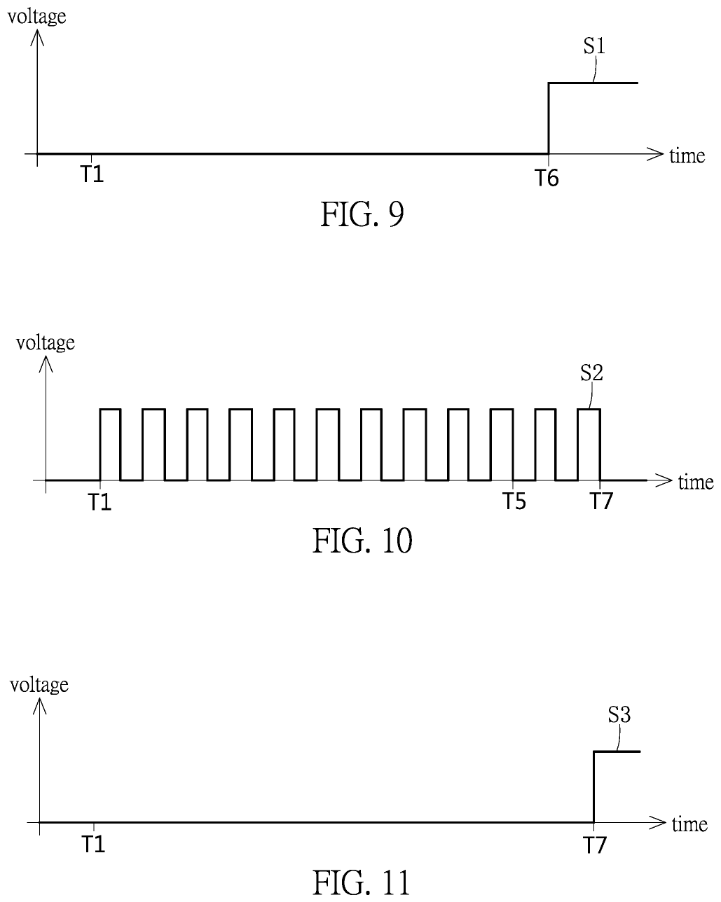

FIG. 9 is a timing diagram of a warning signal of the nebulizer according to one embodiment of the present disclosure. Referring to FIGS. 8 and 9, at a sixth time point T6, the control circuit 5 determines that the aerosol amount index AI of the aerosol path is less than the stop threshold TH2, and the aerosol amount index AI continues to be less than the stop threshold TH2 for the first check time (for example, determined by step S504). At this time, the control circuit 5 outputs a warning signal S1 to the nebulizing module 2 and notifies the nebulizing module 2 to prepare for stopping the nebulizing operation (step S504).

FIG. 10 is a timing diagram of an activating signal of the nebulizer according to one embodiment of the present disclosure. Referring to FIGS. 8 and 10, at the first time point T1, the user inhales the aerosol of the nebulizer 100. At this time, the control circuit 5 sends an activating signal S2 to the nebulizing module 2 for driving the nebulizing module 2 to perform the nebulizing operation. At the fifth time point T5, even if the control circuit 5 determines that the aerosol amount index AI of the aerosol path is less than the stop threshold TH2, the control circuit 5 still sends the activating signal S2 to the nebulizing module 2. When the control circuit 5 determines that the warning signal S1 continues for the second check time at a seventh time point T7 (for example, determined by step S505), the control circuit 5 stops sending the activating signal S2 to the nebulizing module 2. A difference between the seventh time point T7 and the fifth time point T5 is a buffer time for the nebulizing module 2 to continue its operation, and the buffer time is a sum of the first check time and the second check time.

FIG. 11 is timing diagram of a stop signal of the nebulizer according to one embodiment of the present disclosure. Referring to FIG. 11, after the seventh time point T7, the control circuit 5 sends a stop signal S3 to the nebulizing module 2 for stopping the nebulizing operation of the nebulizing module 2.

### Beneficial Effects of the Embodiments

In conclusion, in the aerosol amount detection method and the nebulizer provided by the present disclosure, an aerosol amount can be accurately detected in the second chamber (i.e., an aerosol chamber), and an amount of the medical liquid in the nebulizer can be determined based on the aerosol amount, such that the probability of the nebulizing module continuing to vibrate when the medical liquid is exhausted can be reduced. It should be noted that the aerosol amount detection method and the nebulizer of the present disclosure can also be used simultaneously with an existing detection method for detecting the medical liquid in a medicine cup, thereby increasing a success rate of determining the amount of the medical liquid.

Furthermore, when the reflected light signal received by the light detection circuit is used to detect the aerosol amount in the second chamber (i.e., the aerosol chamber), other aerosol parameters (such as the aerosol particle size or the aerosol concentration) can also be simultaneously obtained. The above-mentioned aerosol parameters can be adjusted by controlling the vibration frequency of the nebulizing module.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An aerosol amount detection method, which is adapted to a nebulizer, the aerosol detection method comprising:
continuously emitting, by a light detection circuit, a plurality of light signals to an aerosol path of the nebulizer for obtaining a plurality of light intensity values;
calculating, by a control circuit, an aerosol amount index of the aerosol path according to the plurality of light intensity values; and
outputting, by the control circuit, a warning signal when the aerosol amount index continues to be less than a stop threshold for a first check time.

2. The aerosol amount detection method according to claim 1, wherein, when the warning signal continues for a second check time, the control circuit stops driving a nebulizing module of the nebulizer.

3. The aerosol amount detection method according to claim 2, further comprising: sending, by the control circuit, an activating signal to the nebulizing module for a buffer time after the aerosol amount index is less than the stop threshold; wherein the buffer time is a sum of the first check time and the second check time.

4. The aerosol amount detection method according to claim 1, further comprising: determining, by the control circuit, whether or not a detection value converted from the light intensity value is greater than a stable threshold before the control circuit calculates the aerosol amount index of the aerosol path according to the plurality of light intensity values; wherein, when the detection value is greater than the stable threshold, the control circuit calculates the aerosol amount index of the aerosol path according to the plurality of light intensity values.

5. The aerosol amount detection method according to claim 4, wherein, after the control circuit calculates the aerosol amount index of the aerosol path according to the plurality of light intensity values, the control circuit determines whether or not a change amount of the aerosol amount index is within a stable interval; wherein, when the change amount is within the stable interval, the aerosol amount index is in a stable state.

6. The aerosol amount detection method according to claim 5, wherein, when the aerosol amount index is in the stable state, the control circuit calculates an average aerosol amount index, and the stop threshold is the average aerosol amount index divided by a stop parameter.

7. The aerosol amount detection method according to claim 1, further comprising: receiving, by the light detection circuit, a plurality of reflected light signals after the light detection circuit continuously emits the plurality of light signals to the aerosol path, and obtaining the light intensity values of the reflected light signals.

8. The aerosol amount detection method according to claim 1, wherein the control circuit calculates at least one aerosol parameter of the aerosol path according to the plurality of light intensity values, and the control circuit controls a vibration frequency of the nebulizing module to adjust the at least one aerosol parameter.

9. A nebulizer, comprising:
a cup configured to store a medical liquid;
a nebulizing module connected to the cup, wherein the nebulizing module is configured to convert the medical liquid into an aerosol and spread the aerosol to an aerosol path;
a host connected to the cup;
a light detection circuit disposed inside the host; and
a control circuit disposed inside the host and connected to the light detection circuit;
wherein the light detection circuit is configured to continuously emit a plurality of light signals to the aerosol path for obtaining a plurality of light intensity values;
wherein the control circuit is configured to calculate an aerosol amount index of the aerosol path according to the plurality of light intensity values;
wherein, when the control circuit determines that the aerosol amount index continues to be less than a stop threshold for a first check time, the control circuit outputs a warning signal.

10. The nebulizer according to claim 9, wherein the control circuit includes an input interface, a central processing unit, a memory, a timer, and an output interface, the central processing unit is electrically connected to the input interface, the memory, the timer, and the output interface, the memory stores a stop threshold, the input interface receives the plurality of light intensity values, and the central processing unit calculates the aerosol amount index; wherein, when the central processing unit determines that the aerosol amount index continues to be less than the stop threshold for the first check time, the central processing unit outputs the warning signal through the output interface.

11. The nebulizer according to claim 9, wherein, when the warning signal continues for a second check time, the control circuit stops driving the nebulizing module.

12. The nebulizer according to claim 11, wherein the control circuit continues to send an activating signal to the nebulizing module for a buffer time after the aerosol amount index is less than the stop threshold, and the buffer time is a sum of the first check time and the second check time.

13. The nebulizer according to claim 9, wherein, when a detection value converted from the light intensity value is greater than a stable threshold, the control circuit calculates the aerosol amount index of the aerosol path according to the plurality of light intensity values.

14. The nebulizer according to claim 13, wherein, when a change amount of the aerosol amount index is within a stable interval, the aerosol amount index is in a stable state.

15. The nebulizer according to claim 14, wherein, when the aerosol amount index is in the stable state, the control circuit calculates an average aerosol amount index, and the stop threshold is the average aerosol amount index divided by a stop parameter.

16. The nebulizer according to claim 9, wherein the light detection circuit receives a plurality of reflected light signals from the aerosol path and obtain the light intensity values of the reflected light signals.

17. The nebulizer according to claim 9, wherein the control circuit calculates at least one aerosol parameter of the aerosol path according to the plurality of light intensity values, and the control circuit controls a vibration frequency of the nebulizing module to adjust the at least one aerosol parameter.
